(19) United States
(12) Patent Application Publication
(10) Pub. No.: US 2006/0035380 A1
(43) Pub. Date: Feb. 16, 2006

(54) Fake-proof marking of a composition

(75) Inventor: Didier Saint-Leger, Clichy (FR)

Correspondence Address: OBLON, SPIVAK, MCCLELLAND, MAIER & NEUSTADT, P.C., 1940 DUKE STREET, ALEXANDRIA, VA, 22314, US

(73) Assignee: L'OREAL, Paris (FR)

(21) Appl. No.: 11/076,879

(22) Filed: Mar. 11, 2005

Related U.S. Application Data
(60) Provisional application No. 60/555,673, filed on Mar. 24, 2004.

(30) Foreign Application Priority Data
Mar. 12, 2004 (FR) 0450512

(51) Int. Cl. G01N 33/24

(52) U.S. Cl. 436/27

(57) ABSTRACT

The invention relates to a process for the fake-proof marking of a product, by introducing at least one non-radioactive stable isotope of at least one atomic element, such that the content of the isotope in the product is higher than its natural content, the isotope being chosen from natural isotopes that have a natural isotopic content of less than 1.2%, and better still less than 1%.

### REFERENCE TO PRIOR APPLICATIONS

This application claims priority to U.S. provisional application 60/555,673 filed Mar. 24, 2004, and to French patent application 0450512 filed Mar. 12, 2004, both incorporated herein by reference.

### FIELD OF THE INVENTION

The invention relates to the fake-proof marking of a composition, for example a manufactured cosmetic or dermatological product. Such marking can be effected by a method comprising, during the manufacture of the product, the enrichment in one or more non-radioactive stable isotopes of an atomic element(s) included in the constitution of the composition.

The invention also relates to the use of one or more non-radioactive stable isotopes enriched in an atomic element as a marker in or for the manufacture of a manufactured composition such as a cosmetic or dermatological product.

As used herein, enrichment in an isotope of an atomic element means that the content of this isotope in the composition is higher than its natural content.

According to the invention, the enriched non-radioactive stable isotope(s) is (are) preferably chosen from natural isotopes having a natural isotopic content of less than 1.2% and better still less than 1%.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Faking (counterfeiting) concerns many industrial sectors, for instance the fragrance industry and pharmaceutical products, and in particular dermatological products and cosmetic products.

Many products are victims of faking, especially when they enjoy commercial success. It is often difficult to combat these attempts at faking, due to lack of means for safely differentiating the original product from the fake product and for ensuring authentication of the original product.

The use of stable non-radioactive isotopes to mark products and processes for authenticating a product by isotopic marking are known in the prior art.

Mention may be made, for example, of document FR 2 673 291, which describes a process for authenticating the origin of a product consisting of organic compounds by isotopic marking with carbon-13 (13C). According to the document, the deliberate modification by adding one or more molecules enriched in 13C causes a desired variation in the isotopic composition of the mixture of organic compounds constituting a product, and especially a product obtained industrially, which makes it possible to constitute a discrete, inimitable, entirely harmless signature (unlike products subjected to radioactive marking) that is detectable only by specialized analytical means, especially by isotopic mass spectrometry.

Mention may also be made of documents U.S. Pat. No. 4,862,143 and U.S. Pat. No. 4,742,340, which describe methods for authenticating articles comprising a non-metallic body (financial documents, banknotes, passports, etc.) and a “label” attached to this body, the label comprising an atomic element enriched in a determined amount of iron-57 (57Fe), europium-151 (151EU) or tin-119 (119Sn).

Osmium-197 (119Os) is also known in document WO 97/46981 for protecting banknotes and valuable documents against forgery.

The abovementioned uses and processes all relate to non-radioactive stable isotopes whose abundance or natural isotopic content is greater than 1%.

| Element | Natural isotopic abundance |
|---|---|
| Carbon-13 | 1.108% |
| Iron-57 | 2.17% |
| Europium-151 | 47.77% |
| Tin-119 | 8.58% |
| Osmium-187 | 1.64% |

Although the natural isotopic content of the abovementioned isotopic elements does not in any way prevent their use, it does, however, have as a consequence, for reasons of detection, the need for a quantitatively large isotopic enrichment, in order to ensure a subsequent irrefutable and safe detection of the isotopic enrichment. This rule, which is comparable to the notion of a signal/noise ratio, which is well known to analytical chemists, entails industrially unacceptable surcharges by greatly increasing the industrial cost price of the finished product. In addition, the isotopes whose abundance or natural isotopic content is greater than 1.2% and in particular greater than 1% cannot be used to mark compositions intended to be used in man or animals, the dosage of which is strict. It is understood, in point of fact, that in order subsequently to be able to detect compositions enriched with isotopes whose abundance or natural isotopic content is greater than 1.2%, or even 1%, the isotopic enrichment is such that the rigorous dosage is not always complied with.

Moreover, the incorporation of large amounts of these isotopes may have the end result of modifying the natural equilibria of the elements under consideration, which may have environmental consequences.

Although non-radioactive stable isotopes with a natural isotopic content of less than 1.2% and in particular less than 1% have been known for a long time, and although faking is a problem that has been known for many years, no industrial process of fake-proofing of a manufactured cosmetic or dermatological product, by isotopic marking with natural isotopes whose natural isotopic content is less than 1.2%, or even 1%, has, to the inventor's knowledge, ever been described.

There is thus still a need for marking a manufactured cosmetic or dermatological product, which are simple to perform, economically acceptable and compatible with the legal and environmental provisions in force.

### DETAILED DESCRIPTION OF THE PREFERRED EMBODIMENTS

The invention thus relates to a process, which can be carried out industrially, for the fake-proof marking of a manufactured compositon, such as a cosmetic or dermatological product, which comprises introducing, during or after manufacture of the composition, at least one non-radioactive stable isotope of at least one atomic element included in its constitution, such that the content of the isotope in the end product is higher than its natural content, the isotope being chosen from natural isotopes that have a low natural isotopic content, i.e. less than 1.2% and better still less than 1%.

One preferred subject of the invention is, more specifically, a process for the fake-proof marking of a manufactured cosmetic or dermatological product, the product comprising a physiologically acceptable medium, which comproses introducing, during or after its manufacture, at least one non-radioactive stable isotope of at least one atomic element included in the constitution of the product, such that the content of the isotope in the manufactured product is higher than its natural content, the isotope being chosen from natural isotopes that have a natural isotopic content of less than 1.2% and better still less than 1%.

For the purposes of the present invention, the term “mark” means the manner of rendering a molecule, a composition, an object, an article or a manufactured cosmetic or dermatological product identifiable, recognizable or distinguishable, relative to a molecule, a composition, an object, an article or a cosmetic or dermatological product, whose origin or source and/or whose authenticity it is desired to know.

For the purposes of the invention, the term “at least one” means one or more (e.g., 1, 2, 3 or more).

The process according to the invention makes it possible, by favouring an enrichment in an isotope (or more than one) of an element naturally present at an abundance of less than 1.2% and better still less than 1%, during or after the manufacture of, e.g., a manufactured cosmetic or dermatological product, to introduce small amounts of the isotope and to make it detectable more easily or more readily under economically acceptable conditions.

For the purposes of the present invention, the term “atomic element” means an atomic element included in the Periodic Table of the chemical elements. For the purposes of the present invention, the term “physiologically acceptable medium” means a medium that is compatible with human keratin materials and/or mucous membranes, and which is non-toxic. This medium may be suitable for topical (or direct) application to the human keratin materials and/or mucous membranes, or for oral application. For the purposes of the present invention, the term “human keratin materials” means the skin, including the scalp, and keratin fibres such as the hair, the eyelashes, the eyebrows, body hairs and the nails, and more especially the hair, of human beings. The term “mucous membranes” mainly means the lips of the face.

The process according to the invention thus has the advantage of being able to provide products (e.g., molecule, composition, object, or cosmetic or dermatological article that may be manufactured) marked according to a “minimum” rule in order to respect as closely as possible the natural equilibria of the chemical elements present in the product and on the planet, especially when it is a matter of a mass-consumption product, while at the same time ensuring the reliability of the subsequent analytical detection by the known analytical means.

In the case of compositions intended for human use (e.g., cosmetic or dermatological compositions), the “minimum” isotopic enrichment according to the process of the invention also has the advantage of being able to provide compositions that can be detected subsequently without their strict dosage being modified. Such is the case, for example, when the dosage (doses to be used) relates to very small amounts of an active agent that is effective at low concentration (hormones, vitamin D, etc.). Thus, for example, supplementary hormone replacement therapy in the case of menopausal women involves the use of transdermal patches in which a hormone (17β-estradiol) diffuses slowly intracutaneously, at very low doses, of from 25 μg to 100 μg over 24 hours. It is thus appropriate, while also respecting the need for low cost, to use the smallest possible amount of isotopic marker. Thus, the addition of 5 ng (nanograms) to 25 μg of 17β-estradiol D3 (16.16.17), reference Isotec 49, 118-7, during the preparation of the patch will be sufficient for a subsequent faking detection without modifying the strict dosage. Such is also the case, for example, for a cosmetic gel capsule into which is incorporated vitamins (A, E, F or C) or an ester thereof (especially ester of vitamin C or F), β-carotene, lycopene or glucose, marked with an isotope having a natural content of less than 1%. For example, the isotope 17O may be incorporated at very low concentrations, without any physiological consequence on the body of the person regularly taking this type of gel capsule (for example for a healthy complexion or to combat natural hair loss), which would not be the case with the use of a natural isotopic element whose content is higher than 1.2%, and in particular 1%, which would require the incorporation into the gel capsule of larger amounts of constituents that are beneficial for the human keratin materials or mucous membranes.

The isotopic marking according to the invention can preferably be performed during the manufacture of the product, via the deliberate introduction, for example known only to the manufacturer, of at least one constituent, starting material or intermediate of the product, certain atom(s) of which have been enriched in non-radioactive stable natural isotopes having a natural isotopic content of less than 1.2% and better still less than 1%. As an alternative, enriched material may be introduced after the product is manufactured, or added both during and after manufacture.

Thus, one or more atoms enriched in natural isotopes will be present in the finished product, unbeknownst to any person other than the manufacturer and/or the legal proprietor of the product, in a slightly higher proportion.

The demonstration and detection of the isotopic enrichment that is achieved according to the invention is performed via well-known analytical means such as mass spectrometry, X-ray fluorescence, SIMS (secondary ion mass spectroscopy) or NMR (nuclear magnetic resonance). The practical protocols to be implemented are, for example, those described in documents WO 00/79569, U.S. Pat. No. 4,862,143 and/or U.S. Pat. No. 4,742,340. As regards the principles for the quantitative determination of the increase in the content of stable isotope, such is within the skill of one of ordinary skill in this art in view of this disclosure, and reference may be made to information made available on the internet by, for example, the Center for Applied Isotope Studies, University of Georgia, currently http://www.uga.edu/˜cais/home.htm, e.g., http://www.uga.edu/˜sisbl/stable.html.

Advantageously, the isotope(s) used in the process of the invention has (have) atomic masses ranging from 2 to 100. They are preferably chosen from deuterium (2D or 2H, hydrogen isotope), helium-3 (3He), carbon-13 (13C), nitrogen-15 (15N), oxygen-17 (17O), oxygen-18 (180), neon-21 (2Ne), sulfur-33 (33S), sulfur-36 (36S), argon-36 (36Ar), argon-38 (38Ar), potassium-40 (40K), calcium-42 (42Ca), calcium-43 (43Ca), calcium-46 (46Ca), calcium-48 (48Ca), iron-58 (58Fe), nickel-64 (64Ni), zinc-70 (70Zn), selenium-74 (74Se), krypton-78 (78Kr) and strontium-84 (84Sr). Very preferably, the non-radioactive stable isotope is chosen from deuterium, nitrogen-15, oxygen-17, oxygen-18, sulfur-36, calcium-46 and carbon-13 (13C).

Advantageously, the enrichment in the non-radioactive stable isotope is reflected in the manufactured product by a ratio: final isotope content/natural content ranging from 1.01 to 10⁶, preferably from 1.02 to 10 000 and very preferably from 1.05 to 5000, including 1.1, 1.25, 1.5, 1.75, 2, 5, 10, 30, 70, 100, 500, 1000, 2000, 3000 and 4000, and all values and subranges between all stated values herein.

According to the invention, the constituent, the starting material or the marked intermediate is, for example, a simple or complex, solid, liquid or gaseous molecule, of organic or mineral origin. It is advantageously:
for fragrances, ethanol or aromatic organic products;
for makeup products (especially nail varnishes), pigments (for example iron oxide or alumina), coalescers (latices) or solvents;
for cosmetic products in general for topical application (especially skincare products and haircare products), fatty substances, water-soluble solvents, preferably ethanol; silicones; hair dyes, preferably a direct dye, or a mineral, organic or nacreous pigment; oxidation dye precursors; couplers or coloration modifiers; surfactants; preserving agents; polymers, preferably a film-forming polymer; hair-relaxing agents or permanent-waving agents; antioxidants, preferably aqueous hydrogen peroxide solution; reducing agents; cosmetic active agents, for instance sunscreens, moisturizers, skin pigmenting agents, astringents, antidandruff agents;
for dermatological creams or ointments, active principles, or excipients,
for cosmetic oral supplements, ingestable active agents such as vitamins, oils of plant origin, carotenoids (lycopene or β-carotene), coating agents.

Many molecules including stable isotopic atoms with a natural isotopic content of less than 1.2% and better still less than 1% are commercially available, in a high enrichment purity. For example, the company Isotec, whose catalogue is available on the Internet (http://www.sigmaaldrich.com), provides more than 2000 organic or mineral molecules within which the isotopic purity (richness of the content of this element in the molecule) may be up to 99%.

The isotopic data concerns only the constitutive state of the core of the stable isotopic atom (neutrons+protons) and in no way its electronic entourage, which characterizes the chemical activity of the atom. It follows therefrom that the intrinsic properties of the molecule in enriched form will be strictly comparable to that of the molecule containing only natural isotopes.

For the purposes of the present invention, the term “product” includes any cosmetic or dermatological molecule, composition, object or article that can be manufactured.

Advantageously, the process according to the invention will provide marked cosmetic or dermatological products, intended to come into contact with human keratin materials (skin, hair, body hairs, eyelashes, eyebrows or nails) or human mucous membranes such as the lips of the face (either by topical application or by ingestion). Very preferably, the product is a cosmetic or dermatological composition for topical (or direct) application.

In the topical-application cosmetic field, the composition for implementing the process according to the invention may especially be in the form of:
a makeup product for facial skin, body skin or the lips, such as a foundation, a tinted cream, a makeup rouge, an eyeshadow, a free or compact powder, a concealer stick, a cover stick, a lipstick, a lipcare product, a nail varnish, a nailcare product, a mascara, a treating mascara or an eyeliner;
an antisun composition or an artificial tanning composition;
an aftershave gel or lotion;
a hair-removing cream;
a dermatological composition;
a solid composition such as a cleansing soap or bar;
an aerosol composition also comprising a propellant under pressure;
a hairsetting lotion or a styling cream or gel,
a dye composition (especially an oxidation dye) optionally in the form of a dye shampoo;
a hair-restructuring lotion, a permanent-waving composition, or a lotion or gel for preventing hair loss;
a haircare composition, especially in the form of a cream;
an antisun gel;
a scalp care composition, especially for preventing hair loss or for hair regrowth;
a washing composition such as a shampoo, a shower gel or a makeup-removing product;
a hair-conditioning composition, especially a detangling composition;
a care, treatment or protective product for keratin materials, and especially for facial skin or body skin, including the scalp, such as a care composition (day composition, night composition or moisturizing composition) for the face or the body; an anti-wrinkle or anti-ageing composition for the face; a matting composition for the face; a composition for irritated skin; a makeup-removing composition; a body milk, especially a moisturizing milk and optionally an after-sun milk.

For oral use, the compositions may be in the form of a powder, a pill, a tablet or a syrup.

The process according to the invention may also consist in introducing one or more molecules not conventionally included in the constitution of the final product, but which will not modify the final intrinsic quality.

The present invention also relates to the use of at least one non-radioactive stable isotope of at least one atomic element as a marker in a manufactured cosmetic product or for the manufacture of a manufactured dermatological product, this cosmetic or dermatological product containing a physiologically acceptable medium, such that the content of the isotope in the manufactured product is higher than its natural content, the isotope being chosen from natural isotopes that have a natural isotopic content of less than 1.2%.

The present invention also relates to the use, especially the cosmetic use, of at least one non-radioactive stable isotope of at least one atomic element as a marker in a manufactured cosmetic product or for the manufacture of a manufactured dermatological product, as an agent for detecting faking or for detecting the faking of the cosmetic or dermatological product, this cosmetic or dermatological product containing a physiologically acceptable medium and the isotope being present in the manufactured product in a content higher than its natural content, the isotope being chosen from natural isotopes that have a natural isotopic content of less than 1.2%.

The present invention also relates to the use of at least one non-radioactive stable isotope of at least one atomic element as a marker in or for the manufacture of a cosmetic or dermatological molecule, object, article or manufactured product, the isotope being chosen from natural isotopes that have a natural isotopic content of less than 1.2% and better still less than 1%, and being present in the molecule, object, article or manufactured product at a content higher than its natural content.

A preferred process according to the invention comprises a) at least one step of manufacture of a manufactured cosmetic or dermatological product, marked by means of an enrichment in at least one non-radioactive stable isotope of at least one atomic element included in the manufacture of the manufactured product (and in particular in one of the molecules or ingredients of the composition of this manufactured product, or alternatively in one of the molecules or ingredients of the packaging containing this composition), the isotope being chosen from natural isotopes that have a natural isotopic content of less than 1.2%, and at least one step of detecting the presence or absence of the enrichment in the non-radioactive stable isotope.

The detection of enriched isotope may be performed in particular by NMR, MS or SIMS analysis.

The process according to the invention may be illustrated especially by means of the following application examples:

1) Fragrancing Cosmetic Composition

The use of ethanol as CD3CD20D (ref. Isotec 18,641-4) or CH3CH2170H (Isotec 58,629-3) or alternatively CH3CH2180H (Isotec 60,987-0) makes it possible to produce marked fragrancing cosmetic compositions. From an analytical viewpoint, the determination, after chromatographic separation of the ethanolic fraction, will show a clear increase in the contents of D, 170 or 180. Two more specific embodiments of fragrancing cosmetic compositions that may be used according to the invention are given below.

Fragrancing cosmetic composition 1 (in %, weight/weight)

| | | |
|---|---|---|
| Fragrancing essence (mixture of natural esters and | 8.00 | g |
| aldehydes) | | |
| Anti-UV screening agent (sold under the name | 1.00 | g |
| Covasorb) | | |
| Butylhydroxytoluene | 0.05 | g |
| Ethanol | 50.00 | g |
| CH3CH218OH (Isotec 60, 987-0) | 0.001 | g |
| Water | qs 100 | g |

Fragrancing cosmetic composition 2 (in %, weight/weight)

| | | |
|---|---|---|
| Fragrancing essence (mixture of natural esters and | 8.00 | g |
| aldehydes) | | |
| Anti-UV screening agent (sold under the name | 1.00 | g |
| Covasorb) | | |
| Butylhydroxytoluene | 0.05 | g |
| Ethanol | 50.00 | g |
| CH3CH217OH | 0.001 | g |
| Water | qs 100 | g |

2) Oil Included in the Manufacture of a Cosmetic Composition

Plant oils are essentially more or less variable mixtures of triglycerides bearing fatty acids of different nature and of different chain length. Among these, Tripalmitine (3 palmitic acids esterified with the 3 hydroxyls of glycerol) is a triglyceride most conventionally encountered.

A producer of plant oils (palm oil, olive oil, sunflower oil, macadamia oil, manoi oil, etc.) may, for example, introduce into his production from 0.01% to 0.05% of glyceryl trihexadecanoate, 16, 16, 16D3 (Isotec 61,547-1), i.e. Tripalmitine in which all the terminal hydrogens of the fatty chains are deuteriums. Mention may also be made of glyceryl trihexadecanoate D31 (Isotec 61,696-6), i.e. Tripalmitine in which all the hydrogens of the fatty chains are deuteriums.

Thus, the analysis of a composition containing an oil naturally containing 5% by weight of Tripalmitine would show, in the case, for example, of an addition of 0.02% of glyceryl trihexadecanoate D31 by weight of the entire oil, a marked increase in the deuterium content, as regards this triglyceride, going from a deuterium content of 0.015% to 0.37% approximately; a technique such as GC-MS would not fail to find this enrichment.

3) Gel Capsule Based on Starch or Gelatin Enriched in Nutrients (Glucose, Vitamins, Carotenoids or Minerals)

In many ingestable cosmetic compositions, the active agent is coated in a hard gel capsule, the weight of which may range, for example, from 500 mg to 1 gram and of which starch (or gelatin) is a major constituent, of from 95% to 99%, the remainder being binders and dyes.

The introduction into the gel capsule paste, before coating the active agent, for example of glucose C6D1206 (Isotec 61,633-8) to a content of 0.001% thereof, allows marking of the ingestable composition, without entailing high industrial costs. The analysis of the gel capsule thus created will be performed by dissolving it, and then isolating the glucose thus released. Conventional NMR or mass spectrometry analytical techniques will make it possible to verify its isotopic composition.

4) Makeup Composition Based on Mineral Pigments

In the preparation of makeup compositions, pigments such as aluminium oxide and ferric oxide are usually introduced at concentrations of from 0.5% to 12%, for example from 0.5% to 4%.

The replacement of 0.02% of aluminium oxide Al2O3 (out of 2% of total aluminium oxide used, for example), during the preparation of the composition, with aluminium oxide Al217O3 (Isotec 60,993-5) in which the 3 oxygen atoms are in the form 17O, leads to a total content of 1.003% of 17O instead of the initial 0.037%, which is readily detectable by mass spectrometry. In an equally comparable manner, iron oxide Fe217O3 (Isotec 58,917-9) may also be selected, by replacing some of the same iron oxide comprising oxygen in natural oxygen-16 form, which is the most abundant form.

A makeup product manufactured with such a pigment will consequently be enriched in these elements and may thus be identified/authenticated by the use of the SIMS method described above.

5) Cosmetic Compositions for the Hair

Hair dye preparation based on 15N2 PPD

Presynthesis of 15N2 para-phenylenediamine (PPD or 1,4-diaminobenzene) by simple reduction of 15N2 para-nitroaniline of which the 2 nitrogen atoms are in 15N form (sold by the company Isotec/Sigma Aldrich, reference 48,773-2).

5 grams of 15N2 para-nitroaniline, 8 g of 5% palladium-on-active charcoal (containing 50% water), 150 ml of 96° ethanol and 150 ml of water are placed in a hydrogenator.

The reduction takes place in half an hour at a hydrogen pressure of about 8 bar and at a temperature that is gradually raised to 75° C.

After filtering off the catalyst (Pd) under nitrogen, the filtrate is evaporated to dryness under reduced pressure.

The crystalline compound obtained is then purified by recrystallization from refluxing ethanol. A para-phenylenediamine in which the two nitrogen groups (NH2) consist of stable nitrogen-15 isotopes is thus obtained.

Dye preparation

| | |
|---|---|
| Para-phenylenediamine | 0.1500 g |
| 15N2 PPD (15N2 1,4-diaminobenzene) | 0.0015 g |
| 1-Amino-4-hydroxybenzene | 0.0500 g |
| 1,3-Dihydroxybenzene | 0.1500 g |
| 1-Hydroxy-3-aminobenzene | 0.1500 g |
| 2-N-methylamino-4-β-hydroxyethyloxynitrobenzene | 0.0500 g |
| tert-Butylhydroquinone | 0.1660 g |
| Sodium bisulfite at 35° Be | 1.5000 g |
| Oleyl alcohol glycerolated with 2 mol of glycerol | 5.0000 g |
| Oleyl alcohol glycerolated with 4 mol of glycerol | 5.0000 g |
| Oleic acid | 5.0000 g |
| Oleic diethanolamide | 5.0000 g |
| Oleic diethanolamide | 12.0000 g |
| Ethyl alcohol | 10.0000 g |
| 2-Ethoxyethanol | 12.0000 g |
| Ethylenediaminetetraacetic acid | 0.2000 g |
| Aqueous ammonia at 22° Be | 10.2000 g |
| Water qs | 100 g |

This composition is a liquid that is mixed at the time of use with its weight of 20-volumes aqueous hydrogen peroxide solution.

Hair-washing composition in the form of a shampoo (in %, weight/weight)

| | |
|---|---|
| Laureth-12 | 0.250 |
| Sodium laureth sulfate | 8.400 |
| D25 sodium lauryl sulfate (Isotec 45,185-1) | 0.001 |
| Sodium methyl paraben | 0.200 |
| Propylene glycol | 0.450 |
| DMDM hydantoin | 0.110 |
| Cocamide MAE | 0.500 |
| Cocobetaine | 3.000 |
| Sodium chloride | 1.650 |
| Fragrance | 0.500 |
| Acid tallow 3 (dye) | 0.002 |
| Water qs | 100 |

Nutritive Hair Formula Based on Glucose 6-octadecenoate

The presynthesis of glucose 6-octadecenoate may be performed according to the synthetic process described in document EP 1 371 658, using 13C18 linoleic acid (all 18 carbon atoms being in 13C form) and 13C6 D glucose (all 6 carbon atoms being in 13C form), the two compounds being commercially available from the company Isotec/Sigma Aldrich. The 24 carbon atoms constituting the ester thus obtained are in the 13C form.

| | | |
|---|---|---|
| 13C24 glucose 6-octadecenoate | 0.5 | g |
| 40 oxyethylenated hydrogenated castor oil | 0.5 | g |
| Butylhydroxytoluene | 0.1 | g |
| Ethanol | 50.7 | g |
| Fragrance qs | | |
| Water qs | 100 | g |

6) Cosmetic skin compositions
Moisturizing facial cream composition (in %, weight/weight)

| | | |
|---|---|---|
| Cetyl alcohol | 7.00 | |
| 12C glycerol | 5.30 | |
| 13C3 glycerol (Isotec 48,947-6) | 2.00 | |
| Mineral oil | 2.00 | |
| PEG-50 stearate | 2.50 | |
| Stearic acid | 0.13 | |
| Glyceryl stearate SE | 2.25 | |
| Isopropyl palmitate | 3.00 | |
| Fragrance | 0.30 | |
| Methyl paraben (preserving agent) | 0.30 | |
| Water qs | 100 | g |

The above written description of the invention provides a manner and process of making and using it such that any person skilled in this art is enabled to make and use the same, this enablement being provided in particular for the subject matter of the appended claims, which make up a part of the original description and including a process for the fake-proof marking of a product containing a physiologically acceptable medium, comprising introducing to said product at least one non-radioactive stable isotope of at least one atomic element included in the product, such that the content of the isotope in the product is higher than its natural content, the isotope(s) being chosen from natural isotopes that have a natural isotopic content of less than 1.2%.

As used above, the phrases “chosen from,” “selected from the group consisting of,” and the like include mixtures of the specified materials.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein.

1. A process for the fake-proof marking of a product, said process comprising introducing to said product at least one non-radioactive stable isotope of at least one atomic element present in the product, in an amount such that the content of the non-radioactive stable isotope in the product is higher than its natural content, the non-radioactive stable isotope(s) being chosen from natural isotopes that have a natural isotopic content of less than 1.2%.

2. The process according to claim 1, wherein at least one of the at least one non-radioactive stable isotope has an atomic mass ranging from 2 to 100.

3. The process according to claim 1, wherein at least one of the at least one non-radioactive stable isotopes is selected from the group consisting of deuterium (2D), helium-3 (3He), carbon-13 (13C), nitrogen-15 (15N), oxygen-17 (17O), oxygen-18 (180), neon-21 (21Ne), sulfur-33 (33S), sulfur-36 (36S), argon-36 (36Ar), argon-38 (38Ar), potassium-40 (40K), calcium-42 (42Ca), calcium-43 (43Ca), calcium-46 (46Ca), calcium-48 (48Ca), iron-58 (58Fe), nickel-64 (64Ni), zinc-70 (70Zn), selenium-74 (74Se), krypton-78 (78Kr) and strontium-84 (84Sr).

4. The process according to claim 1, wherein the at least one non-radioactive stable isotope is at least one of deuterium, nitrogen-15, oxygen-17, oxygen-18, sulfur-36, calcium-46 and carbon-13 (13C).

5. The process according to claim 1, wherein the enrichment in the non-radioactive stable isotope is reflected in the product by a ratio: final isotope content/natural content of 1.01 to 10⁶.

6. The process according to claim 1, wherein the enrichment in the non-radioactive stable isotope is reflected in the product by a ratio: final isotope content/natural content of 1.05 to 5000.

7. The process according to claim 1, wherein the product is a molecule, a composition, an object or an article.

8. The process according to claim 1, wherein the non-radioactive stable isotope(s) are chosen from natural isotopes that have a natural isotopic content of less than 1%.

9. The process according to claim 1, wherein the at least one no radioactive stable isotope comprises carbon-13 (13C).

10. The process according to claim 1, wherein the product is a cosmetic or dermatologic composition comprising a physiologically acceptable medium.

11. The process according to claim 1, wherein the at least one non-radioactive stable isotope is introduced in said product during the preparation thereof.

12. The process according to claim 1, wherein the at least one non-radioactive stable isotope is introduced in said product after the preparation thereof.

13. The process according to claim 1, wherein the at least one non-radioactive stable isotope is introduced in said product both during and after the preparation thereof.

14. The process according to claim 1, further comprising detecting the presence or absence of the content of the non-radioactive stable isotope.

15. A cosmetic or dermatological product, comprising a physiologically acceptable medium and at least one non-radioactive stable isotope in an amount such that the content of the non-radioactive stable isotope in the product is higher than its natural content, the non-radioactive stable isotope(s) being chosen from natural isotopes that have a natural isotopic content of less than 1.2%.

16. The product according to claim 15, the non-radioactive stable isotope(s) being chosen from natural isotopes that have a natural isotopic content of less than 1%.

17. The product according to claim 15, wherein the at least one non-radioactive stable isotope is at least one of deuterium, nitrogen-15, oxygen-17, oxygen-18, sulfur-36, calcium-46 and carbon-13 (13C).

18. The product according to claim 15, wherein the at least one non-radioactive stable isotope comprises carbon-13 (13C).

19. The product according to claim 1, wherein the enrichment in the non-radioactive stable isotope is reflected in the product by a ratio: final isotope content/natural content of 1.05 to 5000.

20. The product according to claim 1, wherein the product is:
a foundation, a tinted cream, a makeup rouge, an eyeshadow, a free or compact powder, a concealer stick, a cover stick, a lipstick, a lipcare product, a nail varnish, a nailcare product, a mascara, a treating mascara, an eyeliner;
an antisun composition or an artificial tanning composition;
an aftershave gel or lotion;
a hair-removing cream;
a dermatological composition;
a solid composition such as a cleansing soap or bar;
an aerosol composition also comprising a propellant under pressure;
a hairsetting lotion or a styling cream or gel,
a dye composition optionally in the form of a dye shampoo;
a hair-restructuring lotion, a permanent-waving composition, or a lotion or gel for preventing hair loss;
a haircare composition;
an antisun gel;
a scalp care composition;
a shampoo, a shower gel or a makeup-removing product;
a hair-conditioning composition; or
a care composition for the face or the body; an anti-wrinkle or anti-ageing composition for the face; a matting composition for the face; a composition for irritated skin; a makeup-removing composition; a body milk.
